(19) United States
(12) Patent Application Publication
(10) Pub. No.: US 2021/0020272 A1
(43) Pub. Date: Jan. 21, 2021

(54) CHARACTERISATION OF AMPORPHOUS CONTENT OF COMPLEX FORMULATIONS BASED ON NON-NEGATIVE MATRIX FACTORISATION

(71) Applicant: Oxford University Innovation Limited, Oxford (GB)

(72) Inventors: Andrew Leslie GODWIN, Oxford (Oxfordshire) (GB); Harold S. GEDDES, Oxford (Oxfordshire) (GB)

(21) Appl. No.: 16/979,890

(22) PCT Filed: Mar. 20, 2019
(86) PCT No.: PCT/GB2019/050781

§ 371 Date: Sep. 11, 2020

(30) Foreign Application Priority Data
Mar. 20, 2018 (GB) 1804427.1

(51) Int. Cl. G16C 20/20; G16C 20/10; G01N 23/20

(52) U.S. Cl. CPC G16C 20/20; G16C 20/10; G01N 2223/605; G01N 2223/304; G01N 2223/345; G01N 23/20

(57) ABSTRACT

Chemical components in a mixture are analysed using scattering data representing the results of a diffraction experiment performed on the mixture. Using non-negative matrix factorisation or another optimisation technique, the scattering data is deconvolved into non-negative basis components that represent contributions to the scattering data from each chemical component and fitting coefficients are derived in respect of the basis components that represent the proportions of chemical components in the mixture.

The present invention relates to the analysis of a mixture of chemical components.

The analysis of mixtures is of interest in general in wide ranging areas of scientific research including development of pharmaceuticals, and there are many available techniques relying on different physical mechanisms. Such techniques each have their own benefits and limitations and the present invention is concerned with providing a new technique that can provide for characterisation of some type of mixtures.

By way of example, there is a particular difficulty in characterising amorphous components in experimental measurements of complex formulations. An example is an amorphous solid dispersion (ASD) which is a mixture wherein the chemical components include a crystalline substance, a polymer binder, and an amorphous form of the crystalline substance. Such ASDs have particular interest in the development of pharmaceuticals, in which case the amorphous substance may be an amorphous pharmaceutical ingredient (API). Amorphous solid dispersions (ASDs) are commonly used to improve the solubility, and hence the bioavailability, of poorly water-soluble molecular pharmaceuticals.

ASDs are highly disordered systems with amorphous active pharmaceutical ingredients (APIs) dispersed in flexible, water-soluble polymers that do not exhibit long-range order. The complex structure, and stability, of ASDs are poorly understood with re-crystallisation of the API the most likely cause of the formulation decomposing. Small amounts of crystallinity can act as a nucleus for re-crystallisation of the amorphous API. Therefore, methods for measuring crystallinity in ASDs could in principle be powerful tools in predicting their stability.

Currently ASDs are studied using a wide range of techniques including mass spectroscopy, nuclear magnetic resonance, and infrared spectroscopy, but with such techniques it remains difficult to quantify the ASD for the purpose of formulating a pharmaceutical. However, methods to quantitatively determine the amount of crystalline API in ASDs remain limited.

According to a first aspect of the present invention, there is provided a method of analysing chemical components in at least one mixture thereof from scattering data representing the results of a diffraction experiment performed on the at least one mixture, the method comprising deconvolving the scattering data into non-negative basis components that represent contributions to the scattering data from the chemical components and deriving fitting coefficients in respect of the basis components that represent the proportions of chemical components in the mixture.

The present method makes use of diffraction to analyse the mixture. In particular the method involves analysis of scattering data representing the results of a diffraction experiment performed on the at least one mixture. The scattering data is deconvolved into basis components. It has been appreciated that by using non-negative basis components in the deconvolution process, it is possible to find basis components that represent contributions to the scattering data from the chemical components in the mixture. This effectively extracts from the scattering data the signal arising from the respective chemical components which may thus be quantified. As such, the deconvolution allows the derivation of fitting coefficients in respect of the basis components that represent the proportions of chemical components in the at least one mixture. This provides information on the proportions of the chemical components that may be difficult to obtain using other techniques, which allows characterisation of the mixture.

By way of example, in the case that the mixture is an ASD wherein the chemical components include a crystalline substance, a polymer binder, and an amorphous form of the crystalline substance, the method provides information on the proportions of the chemical components that may allow the degree of crystallinity in the ASD to be quantified, which is difficult in other techniques.

The deconvolution of the scattering data into non-negative basis components and deriving fitting coefficients in respect of the basis components may be performed using an optimisation technique that optimises the fit of the basis components and the fitting coefficients to the scattering data. Particular advantage is achieved by using non-negative matrix factorisation (NMF) as such an optimisation technique. In that case, further advantage may be achieved by performing the non-negative matrix factorisation using a Metropolis Monte Carlo technique. That allows local minima to be avoided, which might otherwise cause NMF to provide a solution that is not unique.

The deconvolution of the scattering data into non-negative basis components and deriving fitting coefficients in respect of the basis components may be performed applying a constraint on any of the basis components, the fitting coefficients, or a relationship therebetween. This improves the method by allowing account to be taken of known or predictable information about the mixture.

In some cases, the number of chemical components may be known. In that case, the method may be applied to derive the same number of basis components.

In other cases, the number of chemical components may be unknown. In that case, the deconvolution of the scattering data into non-negative basis components may further comprise deriving the number of basis components.

When analysing mixtures of chemical components that have not previously been studied, then at least one, or each, of the basis components may be an unknown function. In that case, the step of deconvolving the scattering data into non-negative basis components may comprise deriving at least one, or each, of the basis components that is an unknown function. This may be achieved by performing the method on scattering data representing the results of a diffraction experiment performed on plural mixtures with different proportions of the chemical components.

When analysing mixtures of chemical components that have previously been studied, then at least one, or each, of the basis components may be a known function derived from the previous studies performed in the past. This allows the method to be performed on instances of the mixture in which the nature of the chemical components is known, but the composition of the mixture is unknown. This may be the situation, for example, when studying mixtures produced during ongoing pharmaceutical production.

Advantageously, the scattering data may comprise a pair distribution function. Alternatively, the scattering data may be of another form, for example comprising a total scattering function.

The method may be applied to scattering data derived from actual performance of a diffraction experiment or may be modelled scattering data derived from a modelled diffraction experiment.

The method has particular application to a mixture that is an ASD wherein the chemical components include a crystalline substance, a polymer binder, and an amorphous form of the crystalline substance.

However, the method may in general be applied to mixtures of any types of chemical components with different diffraction patterns. Some non-limitative examples are described further below.

To allow better understanding, an embodiment of the present invention will now be described by way of non-limitative example with reference to the accompanying drawings, in which:

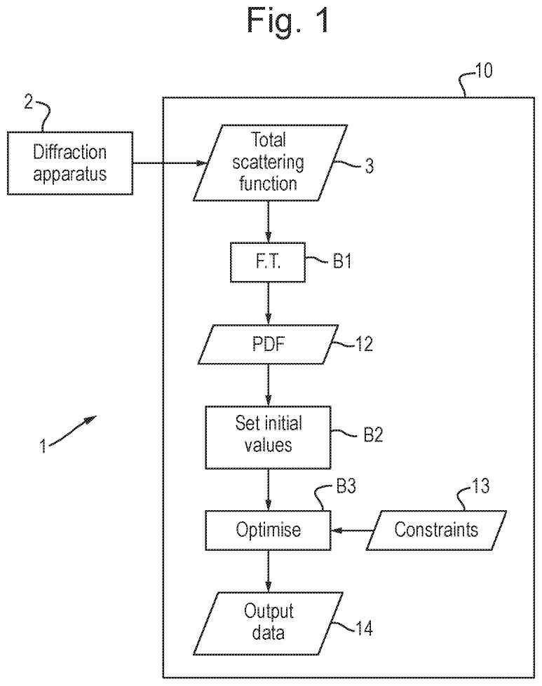

FIG. 1 illustrates a system for analysing a mixture of chemical components;

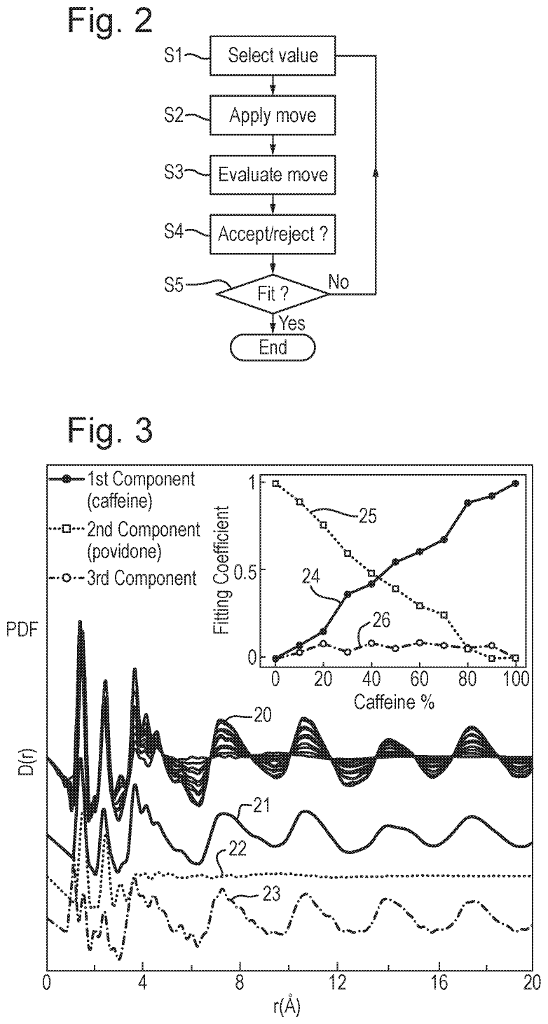

FIG. 2 is a flow chart of a NMF technique;

FIG. 3 is a pair of graphs showing the PDF, basis components and fitting coefficients derived for a physical mixture of caffeine and povidone;

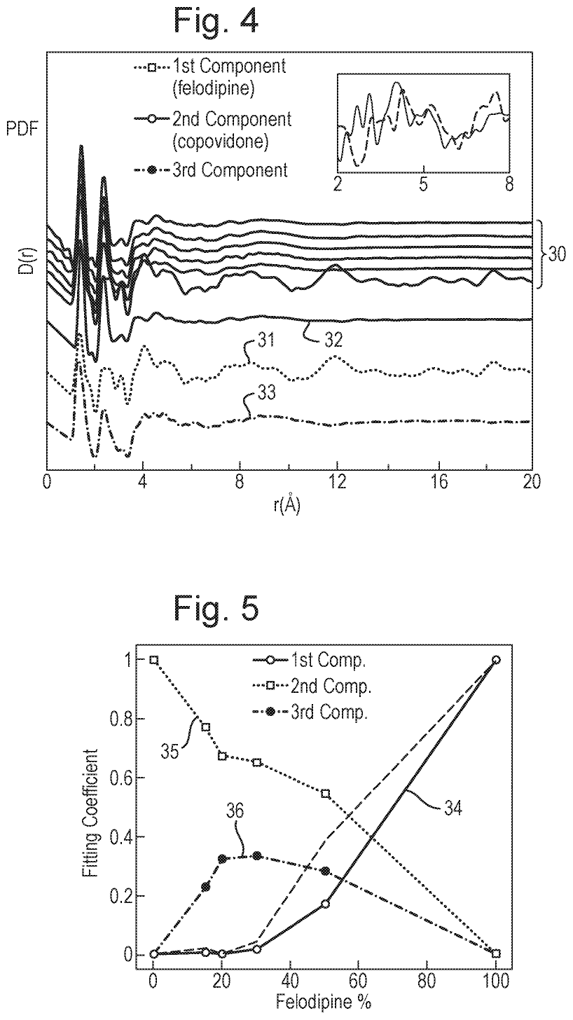

FIG. 4 is a graph showing the PDF and basis components of an ASD of felodipine in copovidone; and

FIG. 5 is a graph showing the fitting coefficients of the ASD of FIG. 4.

A system 1 for analysing a mixture of chemical components is shown in FIG. 1 and arranged as follows.

The system 1 includes a diffraction apparatus 2 that is used to perform a diffraction experiment on samples that each comprise a mixture of chemical components. The diffraction apparatus 2 may be of any conventional type and may include the following features.

The experiment may involve making scattering measurements on samples using a source of radiation. Common sources that may be used include X-rays, neutrons, and/or electrons. Each source of radiation is characterised by a wavelength or range of wavelengths. Typically this wavelength or range of wavelengths is chosen to be small with respect to the usual interatomic distances in materials so as to give good resolution to the pair distribution function. This corresponds also to a maximum value of the scattering vector magnitude Qₘₐₓ, which is typically 20-30 Å⁻¹ for X-ray total scattering measurements.

A typical scattering measurement that may be made involves an apparatus (diffractometer) consisting primarily of a source of radiation, a sample holder/container, and a scattering detector. The scattering geometry (relative orientations/positions of these three components) may be a variable property of the scattering measurement. For a given geometry, the scattering from the sample contained within the sample holder is measured using the detector. Detectors may be energy discriminating or energy integrating.

The detected scattering pattern is often a complex function of various factors including scattering angle, incident radiation energy, and (in some cases) time of flight. The final component of scattering pattern measurement is the normalisation of the scattering function into a total scattering function S(Q) that is only a function of the scattering vector magnitude Q.

The nature of the mixture of chemical components is discussed further below. However, in an illustrative example that is referenced in this description of the system 1, the mixture is an ASD of felodipine and a copovidone polymer. Felodipine is a crystalline substance which constitutes a first chemical component. Copovidone is a polymer binder which constitutes a second chemical component. Within the ASD there also forms an amorphous form of felodipine which therefore constitutes a third chemical component. In general terms, the amount of the crystalline form of felodipine varies with various parameters and is difficult to predict.

On performance of the diffraction experiment on a sample, the diffraction apparatus 2 produces total scattering data 3 representing a total scattering function. Such a total scattering function is the direct result of the diffraction experiment. The total scattering data 3 is supplied to an analysis system 10 which analyses the total scattering data 3.

The diffraction apparatus 2 may be used to perform the diffraction experiment on plural samples that comprise respective mixtures of the same chemical components but with different proportions of the chemical components. This is appropriate to study mixtures of chemical components that have not previously been studied and to derive basis components (described in more detail below) in respect of those chemical components. However, where the basis components are already known, the diffraction apparatus 2 may be used to perform the diffraction experiment on a single sample that comprises a mixture of chemical components (or on plural samples that each comprise a mixtures of the same chemical components in nominally the same proportions to reduce experimental error).

FIG. 1 illustrates functional blocks of the analysis system that carry out the steps of the method performed by the analysis as described further below.

The analysis system 10 may be implemented by a computer apparatus. In this case, a computer program capable of execution by the computer apparatus is provided. The computer program is configured so that, on execution, it causes the computer apparatus to perform the method.

The computer apparatus, where used, may be any type of computer system but is typically of conventional construction. The computer program may be written in any suitable programming language. The computer program may be stored on a computer-readable storage medium, which may be of any type, for example: a recording medium which is insertable into a drive of the computing system and which may store information magnetically, optically or opto-magnetically; a fixed recording medium of the computer system such as a hard drive; or a computer memory.

In block B1, the analysis system 10 performs a Fourier Transform of the total scattering data 3 to derive PDF data 12 that is scattering data that represents a pair distribution function (PDF). The PDF data 12 may be any representation and any normalisation. A PDF is a known representation of the results of a diffraction experiment. A PDF is a useful representation for analysis because it may be considered as describing the distribution of distances between pairs of particles in the sample on which the diffraction experiment is performed. As the PDF is derived from the total scattering function, no information about the sample is lost.

In general terms, PDF analysis is suitable for studying disordered systems such as ASDs, despite their lack of long-range order. The PDF is essentially a histogram of all the interatomic distances in a material and is sensitive to both long and short-range correlations in a material. PDF studies of molecular systems include PDF analysis of crystalline pharmaceuticals and using PDF as a tool for fingerprinting amorphous and nanocrystalline APIs. The majority of PDF studies of amorphous pharmaceuticals have focussed on single-phase amorphous APIs with no polymer in the system.

The remainder of the method performed by the analysis system 10 is performed on the PDF data 12. That said, as the total scattering data 3 is scattering data that is another representation of the results of a diffraction experiment which is a linear function of the PDF data 12, as an alternative, the block S1 could be omitted so that the remainder of the method is performed on the total scattering data 3 instead of the PDF data 12. In general, the methods disclosed herein could equally be performed on any scattering data that is a representation of the results of a diffraction experiment, including linear functions of the total scattering data 3

The PDF data 12 is assembled into an n×m matrix D, where n is the number of data sets corresponding to the number of samples and m is the number of data points in each set.

Although in this example the PDF data 12 is derived from the performance of a diffraction experiment in the diffraction apparatus 2, as an alternative the PDF data 12 may be calculated from a computational model of the mixture.

The analysis system 10 may optionally also store and use constraint data 13 representing a constraint that may be applied in the method as described further below.

In overview, the method performed by the analysis system 10 deconvolves the PDF data 12 into non-negative basis components and derives fitting coefficients in respect of the basis components. This is performed by an optimisation technique implemented in block B3. As discussed below, the non-negative basis components represent contributions to the PDF data 12 from each chemical component and the fitting coefficients represent the proportions of chemical components in the mixture. The basis components comprise an n×k matrix H, and the fitting coefficients make up a k×m matrix W, where k is the number of basis components and n and m correspond to the dimensions of matrix D i.e. the PDF data 12.

As a preliminary step performed in block B2, the analysis system 10 sets initial values for the for the basis components and the fitting coefficients. This may be done in any suitable manner. The initial values may be determined randomly, may be based on prior knowledge, or may be generated in any other way.

By way of example, in illustrative example referenced above, in block B2 the fitting coefficients may initialised to expected values selected to reduce the time taken for convergence. For example, for a sample with 30% felodipine, the felodipine and copovidone basis components given initial fitting coefficients of 0.3 and 0.7 respectively and all other basis components may be initialised as zero.

In block B3, the analysis system 10 performs the optimisation technique to optimise the fit of the basis components and the fitting coefficients to the PDF data 12. In general terms, the optimisation technique processes the refineable parameters, which in this case are the basis components and the fitting coefficients, and refines them against input data, which in this case is the PDF data 12.

By way of example, in illustrative example referenced above, there are three basis components, two of the basis components corresponding to each end-member (i.e. felodipine and copovidone) and the third corresponding to that of the amorphous felodipine.

The optimisation technique optimises the fit of the basis components and the fitting coefficients to the PDF data 12. Typically this involves an iterative process of varying the basis components and/or the fitting coefficients in order to maximise quality of fit between input PDF data 12 and calculated PDF data derived from the basis components and the fitting coefficients.

The optimisation technique performed in block B3 may advantageously be non-negative matrix factorisation (NMF). NMF is in itself one of many known approaches to processing complex data sets and is conceptually related to principal component analysis (PCA). The common idea between NMF and PCA is to deconvolve a large data set into weighted contributions of a small number of basis components. However, in the present case, NMF provides the advantage that each component is directly interpretable in its own right because the basis components are non-negative. That is not the case for PCA where the basis components inevitable include negative values, and so do not correspond any chemical component. As an example, a data set might contain X-ray diffraction patterns of a series of binary mixtures with different mass fractions. PCA analysis would give two basis components, namely the average diffraction pattern, and the difference function for the two elements of the mixture. In contrast, NMF would yield the individual diffraction patterns of the two elements, which is much more useful because each of the basis components corresponds to a chemical component.

Further advantage may be achieved by performing the NMF using a Metropolis Monte Carlo technique. Such performance of NMF using a Metropolis Monte Carlo technique may be referred to as a Metropolis Matrix Factorisation (MMF) technique. This allows local minima to be avoided, which might otherwise cause NMF to provide a solution that is not unique, thereby providing robustness.

NMF is typically carried out using one or a combination of three algorithms. All of these algorithms involve processes that affect all elements of the matrices involved in each step. This means that the same starting configurations will always yield the same result and different starting configurations may yield different results. The main difference between these and the MMF technique is the application of the Metropolis algorithm (sometimes accepting moves worsen the goodness of fit). This means that the same result will always be found, regardless of the starting configuration.

In the MMF technique, elements of matrices W and H are varied by small increments to better approximate D≈WH=A. The goodness of fit is given by the agreement between the matrix D which is the input data, i.e. the PDF scattering data 12, and the product of the multiplication of matrices W and H (D≈WH=A).

By way of example, the NMF technique may be applied by using the method shown in FIG. 2 and performed as follows.

In step S1, a value to change is randomly selected. For example, the value to change that is selected may be an element of W or H, i.e. a fitting coefficient or a value of a basis component.

In step S2, a random move (positive or negative) is applied to the value of the selected element. Counter changes are applied in associated elements.

In step S3, the move is evaluated to determine if the fit of D to WH is improved. In step S4, it is decided whether to accept or reject the move applied in step S2. If the evaluation in step S3 was that the move improved the fit, then the move is accepted. If the evaluation in step S3 was that the move worsened the fit, then the move is accepted with a probability P given by P=exp(−Δ|A−D|²/T) where T is a parameter analogous to temperature.

In step S5, it is decided whether the fit is sufficiently good. If step S5 determines that the fit is not sufficiently good, then the method reverts to step S1. If step S5 determines that the fit is sufficiently good, then the method ends.

Although the NMF and MMF techniques are advantageous, in general other optimisation techniques may be applied in block B3. By way of non-limitative example, other suitable optimisation techniques include least squares minimisation, genetic algorithms, and/or other stochastic optimisation approaches.

Optionally in block B3 further information characterising the mixture may be derived from the basis components or the fitting coefficients. For example, in the case that the chemical components include a crystalline component and an amorphous component, the ratio of amorphous to crystalline components may used to derive a measure of the degree of crystallinity in the dispersion.

Optionally, the analysis system 10 may use the constraint data 13 as follows. The constraint data 13 represents a constraint on the refineable parameters, i.e. the basis components or the fitting coefficients or both, or a relationship between any of the refineable parameters. Then the optimisation technique performed in block B3 is performed applying the constraint. This improves the method by allowing account to be taken of known or predictable information about the mixture and/or making the interpretation of the outputs more intuitive. By way of example, one or more basis components may be fixed to be a known basis component in respect of one or more of the chemical components. This may be applicable where a basis component is known from previous studies. In another example, the sum of the basis components may be fixed to equal unity. In general, the use of constraints provides versatility.

The block B3 outputs output data 14 that includes the derived basis components and fitting coefficients. The output data 14 may optionally also include any or all of: the uncertainties in the derived basis components; the uncertainties in the derived fitting coefficients; parameter covariances of the fit of the derived basis components and fitting coefficients to the PDF data 12; the statistical quality of the fit, and any further information characterising the mixture that is derived in block B3.

It has been appreciated that by using non-negative basis components in the deconvolution performed in block B3, it is possible to find basis components that represent contributions to the PDF data 12 from the chemical components in the mixture. This effectively extracts, from the PDF data 12, the signal arising from the respective chemical components which may thus be quantified. As such, the deconvolution derived fitting coefficients represent the proportions of chemical components in the at least one mixture. This provides information on the proportions of the chemical components that may be difficult to obtain using other techniques, which allows characterisation of the mixture.

In some types of mixture, the number of chemical components may be known, for example from previous studies or other scientific knowledge. In that case, block B3 derives the same number of basis components as chemical components.

In other types of mixture, the number of chemical components may be unknown. In that case, block B3 also derives the number of basis components. This may be achieved by block B3 repeating the method for representations having different numbers of basis components, assessing the statistical significance of the basis parameters in each representation, and selecting one of the representations a particular number of basis components where the statistical significance of each basis component is above a predetermined threshold.

The implementation is robust, straightforward, quantitative, and computationally inexpensive. The quantification is also more meaningful than correlation length analysis.

The nature of the mixture of chemical components will now be considered.

The techniques described herein have particular advantage when the mixture is an amorphous solid dispersion and the chemical components include an amorphous substance, a polymer binder, and a crystalline form of the amorphous substance. This has been established using two model systems, one being a physical mixture of caffeine and povidone, and the other is being a series of felodipine and copovidone ASDs. In both cases, X-ray total scattering data was extracted and PDF data was derived therefrom. As described further below, the physical mixture was well described in terms of the two individual components (caffeine and povidone) and the ASDs required three components (copovidone, crystalline felodipine, and amorphous felodipine). The ratio of amorphous to crystalline felodipine in each sample quantified the degree of crystallinity. 30% loading was determined as the maximum fraction of the API in the ASD, which reflects the current industry understanding.

Thus, it is possible to provide quantification of degree of crystallinity of amorphous solid dispersions, e.g. as a function of API loading in an ASD, or temperature, or humidity. This will help identify maximum loading fractions, monitor crystallisation of APIs, and may play a role in quality control. Structural ‘fingerprinting’ of APIs, including quantitative similarity matching.

However, the mixtures of any types of chemical components in which diffraction provides different diffraction patterns, because that allows the deconvolution into basis functions to be performed. It is particularly suitable for studying complex formulations, which may be difficult to study by other techniques. The mixture may be for example a catalyst support.

Some non-limitative examples of mixtures that may be studied are as follows.

In the simple case, the mixture may comprise chemical components that are different substances.

Where one or more of the chemical components is a polymer, it may be in general be any polymer, for example polyvinylpyrrolidone (povidone, PVP), copovidone (a vinylpyrrolidone-vinyl acetate copolymer), Eudragit (polymethacrylate polymers), hydroxypropyl methylcellulose (HPMC), hypromellose phthalate (HPMCP), polyethylene glycol (PEG), polyacrylic acid, or poloxamer.

Where one or more of the chemical components is a crystalline substance, it may in general any crystalline substance, for example felodipine, caffeine, or nifedipine.

Where one or more of the chemical components is an amorphous substance, it may in general any amorphous substance, which may be an amorphous form of the same substance as another chemical component in the mixture that is a crystalline substance. Examples of substances that may exist in both crystalline and amorphous forms that may be used include felodipine, nifedipine and sulfamerazine.

The mixture may comprise chemical components that include two or more polymorphs of a substance. Polymorphism is the ability of a solid material to exist in more than one form or crystal structure. Ritonavir is an example of a drug known to exist in multiple polymorphs.

The mixture may comprise chemical components that include first and second chemical components and a third chemical component that is an interface or complex between the first and second chemical components. By way of example, with reference to the Haber process in which gaseous nitrogen is adsorbed onto a catalyst (e.g. osmium or iron promoted with K₂O, CaO, SiO₂ and Al₂O₃), the mixture may comprise first and second chemical components that comprise gaseous nitrogen and the catalyst, respectively, and a third chemical component that comprises an interface of nitrogen adsorbed onto the catalyst.

The mixture may comprise chemical components that include plural phases of a chemical entity. For example, the mixture may comprise chemical components that are plural phases in a process, for example an initial phase, a final phase, and one or more intermediate phases. By way of example, with reference to crystallisation of Zeolitic imidazolate framework-8 (ZIF-8) from solution which involves amorphous nuclei (solution phase-amorphous nuclei-crystalline ZIF-8), the mixture may comprise a first chemical component that comprises the solution, a second component that comprises crystalline ZIF-8 and a third component that comprises intermediate amorphous nuclei.

The mixture may comprise chemical components that include a first chemical component that is one or more reactants in a reaction, a second chemical component that comprises one or more products in the reaction, and a third chemical component that comprises one or more intermediates in the reaction. By way of example, reference is made to the phase transformation of LiFePO₄ to FePO₄. In this process the delithiation proceeds via the formation of an intermediate solid solution phase LiₓFePO₄. In this case, the mixture may comprise a first chemical component that comprises the LiFePO₄ phase, a second chemical component that comprises the FePO₄ phase, and a third chemical component that comprises the intermediate LiₓFePO₄.

When analysing mixtures of chemical components that have not previously been studied, then at least one, or each, of the basis components may be an unknown function. In that case, the step of deconvolving the data into non-negative basis components may comprise deriving the at least one, or each, of the basis components that is an unknown function. This may be achieved by performing the method on data representing the results of a diffraction experiment performed on plural mixtures with different proportions of the chemical components, as mentioned above.

When analysing mixtures of chemical components that have previously been studied, then at least one, or each, of the basis components may be a known function derived from the previous studies performed in the past. This allows the method to be performed on instances of the mixture in which the nature of the chemical components is known, but the composition of the mixture is unknown. This may be the situation, for example, when studying mixtures produced during ongoing pharmaceutical production.

There will now be provided further information on the two model systems that were studied, as set out above. Herein, diffraction experiments were performed using a laboratory diffractometer equipped with a Mo X-ray tube (λ=0.71 Å, Qmax=17 Å⁻¹).

The first model system was plural physical mixtures of caffeine and povidone in different proportions, for which data was obtained and analysed as described above. No prior assumption is made of the structure of the system over any length scale.

In FIG. 3, the main graph shows the PDFs 20 of the mixtures and the three basis components 21-23 derived therefrom, whereas the inset shows the fitting components 24-26 respectively derived from the three basis components 21-23. The fitting coefficients 24, 25 of the first and second basis components 21, 22 are as expected for a two-phase system and are similar if only two basis components are deconvolved. Importantly, the third basis component 23 has a relatively small significance (<8%) and fluctuates little for each composition, this indicating a minor contribution of component three for the simple system. Indeed, mixing of caffeine and povidone does little to affect the packing of either end-member. Thus, it is concluded that the first and second basis components 21, 22 correspond to the chemical components caffeine and povidone, and that there is no loss of information in performing the method to deconvolve the PDF into the first and second basis components 21, 22 only.

The second model system was an ASD of felodipine in copovidone in different proportions (15%, 20%, 30% and 50%) prepared by hot melt extrusion, for which scattering data was obtained and analysed as described above. Felodipine is a Ca²⁺ antagonist used to treat hypertension. The copovidone, in which the felodipine is suspended, is in a water-soluble polymer matrix used as a binder.

FIG. 4 shows the PDFs 30 of the ASDs and the three basis components 31-33 derived therefrom, whereas FIG. 5 shows the fitting components 34-36 respectively derived from the three basis components 31-33. In this example, the first and second basis components 31, 32 correspond to the chemical components crystalline felodipine and copovidone. Thus, in this example, the analysis was performed constraining the first and second basis components 31, 32 to the known PDFs of crystalline felodipine and copovidone, whereas the third basis component was allowed to vary.

There are clear similarities between each of the PDFs of the felodipine copovidone ASDs, particularly at low-r (<6 Å). Peaks in the PDF out to nearly 8 Å correspond to the intramolecular interactions of felodipine. There are no significant peaks>8 Å, indicating that there is little ordering of the polymer over distances greater than this (a few monomer units). Additionally, direct indexing of peaks in the PDFs of the ASDs is complex as the similarity of the short-range (intramolecular) structures of the drug and the polymer leads to many different intramolecular separations contributing to the first few peaks in the PDF. For molecular systems the PDF can be thought of in terms of the intramolecular distances at low-r and the intermolecular distances at high-r, with a region of overlap of the two regions. This model becomes more complex for larger molecular systems or multicomponent systems as the regions become more difficult to distinguish due to intramolecular interactions existing over a longer length scale.

As shown in FIG. 5 and similarly to the physical mixtures of caffeine and povidone, the fitting coefficient of the second basis component corresponding to the polymer binder copovidone follows the trend expected for a two-phase system.

However, the fitting coefficients for the first and third basis components corresponding to crystalline and amorphous forms of felodipine show more complex interactions. In particular, the fitting coefficient of the first basis component corresponding to crystalline felodipine has essentially zero contribution to the PDFs of the ASDs containing less than 30% felodipine. Instead, for low loading levels of felodipine, the weights of the third basis component corresponding to amorphous felodipine are comparable to what we might expect for the drug component. In other words, the PDF of crystalline felodipine does not explain the features of the ASDs with less than 30% loadings and instead the free basis component explains the API contribution to the PDFs. For samples with greater felodipine loading, the crystalline drug component becomes increasingly significant, and likewise the fitting coefficient for the third basis component decreases, indicating the presence of crystalline felodipine in the ASDs at greater loading levels.

The third basis component is largely similar to the PDF of crystalline felodipine, particularly in the low-r region. However, the third basis component is convoluted as it describes interactions present in the ASDs that do not exist in either end-member, for example drug-polymer interactions and differences in the packing structure of the two components in the ASDs compared to in their pure forms. Many of these interactions exist over a similar length-range to the intermolecular separations in felodipine and copovidone so isolating specific features is challenging.

Thus, this study identified features in PDFs of the ASDs that cannot be explained by straightforward linear combinations of either end-member. The negligible contribution of the felodipine PDF to describe ASDs with low drug loading levels (likewise the relatively large contribution to third basis component for these samples) indicates structural differences in the ASDs that are not seen in the pure end-members. This indicates a possible molecular reorientation of felodipine from known crystalline polymorphs.

Additionally, the ratio of the fitting coefficient of the first basis component to the fitting coefficient of the third basis component gives the crystallinity factor, which is a useful quantification of the crystallinity in a sample. Established methods exist for quantifying crystallinity in ASDs, but methods from PDF data have not been reported to date. For this series of ASDs, this analysis indicates essentially zero crystallinity until there is greater than 30% felodipine.

The work leading to this invention has received funding from the European Research Council under the European Union's Seventh Framework Programme (FP7/2007-2013)/ERC grant agreement no 279705.

1. A method of analysing chemical components in at least one mixture thereof from scattering data representing the results of a diffraction experiment performed on the at least one mixture, the method comprising deconvolving the scattering data into non-negative basis components that represent contributions to the scattering data from each chemical component and deriving fitting coefficients in respect of the basis components that represent the proportions of chemical components in the mixture.

2. A method according to claim 1, wherein the step of deconvolving the scattering data into non-negative basis components and deriving fitting coefficients in respect of the basis components is performed using an optimisation technique that optimises the fit of the basis components and the fitting coefficients to the scattering data.

3. A method according to claim 2, wherein the optimisation technique comprises non-negative matrix factorisation.

4. A method according to claim 3, wherein the non-negative matrix factorisation is performed using a Metropolis Monte Carlo technique to avoid local minima.

5. A method according to claim 2, wherein the step of deconvolving the scattering data into non-negative basis components and deriving fitting coefficients in respect of the basis components is performed applying a constraint on any of the basis components, the fitting coefficients, or a relationship therebetween.

6. A method according to claim 2, wherein the number of chemical components is unknown and wherein the step of deconvolving the scattering data into non-negative basis components further comprises deriving the number of basis components.

7. A method according to claim 1, wherein at least one of the basis components is an unknown function and the step of deconvolving the scattering data into non-negative basis components comprises deriving the at least one of the basis components that is an unknown function.

8. A method according to claim 7, wherein each of the basis components is an unknown function.

9. A method according to claim 1, wherein the at least one mixture comprises plural mixtures with different proportions of the chemical components.

10. A method according to claim 1, wherein at least one of the basis components is a known function.

11. A method according to claim 10, wherein each of the basis components is a known function.

12. A method according to claim 1, wherein the scattering data represents a pair distribution function.

13. A method according to claim 1, further comprising performing a diffraction experiment and deriving the scattering data therefrom.

14. A method according to claim 1, wherein the mixture is an amorphous solid dispersion and the chemical components include a crystalline substance, a polymer binder, and an amorphous form of the crystalline substance.

15. A method according to claim 1, wherein the chemical components include two or more polymorphs of a substance.

16. A method according to claim 1, wherein the chemical components include first and second chemical components and a third chemical component that is an interface or complex between the first and second chemical components, or wherein the chemical components include plural phases of a chemical entity, or both.

17. (canceled)

18. A method according to claim 1, wherein the chemical components include a first chemical components that is one or more reactants in a reaction, a second chemical component that is one or more products in the reaction, and a third chemical component that is one or more intermediates in the reaction.

19. A computer program capable of execution by a computer apparatus and configured, of execution, to cause the computer apparatus to perform a method according to claim 1.

20. A computer-readable storage medium storing a computer program according to claim 19.

21. An analysis system for determining proportions of chemical components in a mixture from scattering data representing the results of a diffraction experiment performed on the mixture, the analysis system being arranged to deconvolve the scattering data into non-negative basis components that represent contributions to the scattering data from each chemical component and deriving fitting coefficients in respect of the basis components that represent the proportions of chemical components in the mixture.
